(19) United States
(12) Patent Application Publication
(10) Pub. No.: US 2024/0180578 A1
(43) Pub. Date: Jun. 6, 2024

(54) ULTRASONIC SURGICAL INSTRUMENTS AND SYSTEMS INCORPORATING ENHANCED GRASPING FUNCTIONALITY

(71) Applicant: Covidien LP, Mansfield, MA (US)

(72) Inventors: Michael B. Lyons, Boulder, CO (US); David J. Van Tol, Boulder, CO (US)

(21) Appl. No.: 17/799,179

(22) PCT Filed: Mar. 8, 2022
(86) PCT No.: PCT/IB2022/052038

§ 371 Date: Aug. 11, 2022

Related U.S. Application Data
(60) Provisional application No. 63/162,393, filed on Mar. 17, 2021.

(51) Int. Cl. A61B 17/32

(52) U.S. Cl. CPC A61B 17/320092; A61B 2017/320078; A61B 2017/320094

(57) ABSTRACT

An ultrasonic surgical instrument includes an ultrasonic waveguide defining a blade at a distal end thereof, and a jaw member. The ultrasonic waveguide is configured to transmit ultrasonic energy therealong to the blade for treating tissue therewith. The jaw member is movable relative to the blade from a spaced-apart position to an approximated position. The jaw member has a more-rigid structural body and a more-compliant jaw liner engaged with the structural body. The jaw liner defines a tissue grasping surface having a configuration at least partially complementary to a configuration of a tissue grasping surface of the blade such that, in the approximated position, the tissue grasping surfaces are configured to grasp tissue therebetween with the at least partially complementary configurations of the first and second tissue grasping surfaces facilitating tissue grasping.

### CROSS-REFERENCE TO RELATED APPLICATIONS

This application is a 371 National Stage Application of International Application No. PCT/IB2022/052038, filed Mar. 8, 2022, which claims benefit of U.S. Provisional Patent Application No. 63/162,393, filed Mar. 17, 2021, the entire contents of each of which is hereby incorporated herein by reference.

### FIELD

The present disclosure relates to energy based surgical instruments and, more particularly, to ultrasonic surgical instruments and systems incorporating enhanced grasping functionality.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade.

### SUMMARY

As used herein, the term “distal” refers to the portion that is described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is an ultrasonic surgical instrument including an ultrasonic waveguide defining a blade at a distal end thereof, and a jaw member. The blade defines a first tissue grasping surface. The ultrasonic waveguide is configured to transmit ultrasonic energy therealong to the blade for treating tissue therewith. The jaw member is movable relative to the blade from a spaced-apart position to an approximated position. The jaw member has a more-rigid structural body and a more-compliant jaw liner engaged with the structural body. The jaw liner defines a second tissue grasping surface having a configuration at least partially complementary to a configuration of the first tissue grasping surface. In the approximated position, the first and second tissue grasping surfaces are configured to grasp tissue therebetween. The at least partially complementary configurations of the first and second tissue grasping surfaces facilitate the tissue grasping.

In an aspect of the present disclosure, the first and second tissue grasping surfaces define complementary wave-shaped configurations. In such aspects, the first and second tissue grasping surfaces may define complementary wave-shaped configurations extending longitudinally along portions of the blade and the jaw liner, respectively. Alternatively or additionally, the first and second tissue grasping surfaces define complementary wave-shaped configurations extending transversely across portions of the blade and the jaw liner, respectively.

In another aspect of the present disclosure, the first and second tissue grasping surfaces define complementary sine wave-shaped configurations.

In still another aspect of the present disclosure, the first and second tissue grasping surfaces are configured to at least partially interfit with one another in the approximated position.

In yet another aspect of the present disclosure, the ultrasonic surgical instrument further includes an ultrasonic transducer coupled to a proximal portion of the waveguide and configured to produce the ultrasonic energy for transmission along the waveguide to the blade. A housing may support the ultrasonic transducer.

In still yet another aspect of the present disclosure, the ultrasonic surgical instrument further includes a support member and a drive member extending distally from the housing. The waveguide, in such aspects, extends through at least one of the support member or the drive member such that the blade extends distally therefrom. The jaw member is pivotably supported by the support member and operably coupled to the drive member such that translation of the drive member pivots the jaw member between the spaced-apart and approximated positions.

In another aspect of the present disclosure, the ultrasonic surgical instrument further includes a trigger extending from the housing and operably coupled to the drive member such that actuation of the trigger translates the drive member.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 illustrates a surgical system provided in accordance with the present disclosure including an ultrasonic surgical instrument and a surgical generator;

FIG. 2 is a schematic illustration of a robotic surgical system provided in accordance with the present disclosure;

FIG. 3 is an enlarged, longitudinal, cross-sectional view of a distal portion of an end effector assembly configured for use with the ultrasonic surgical instrument of FIG. 1, the robotic surgical system of FIG. 2, or any other suitable surgical instrument or system; and

FIG. 4 is an enlarged, transverse, cross-sectional view of another end effector assembly configured for use with the ultrasonic surgical instrument of FIG. 1, the robotic surgical system of FIG. 2, or any other suitable surgical instrument or system.

### DETAILED DESCRIPTION

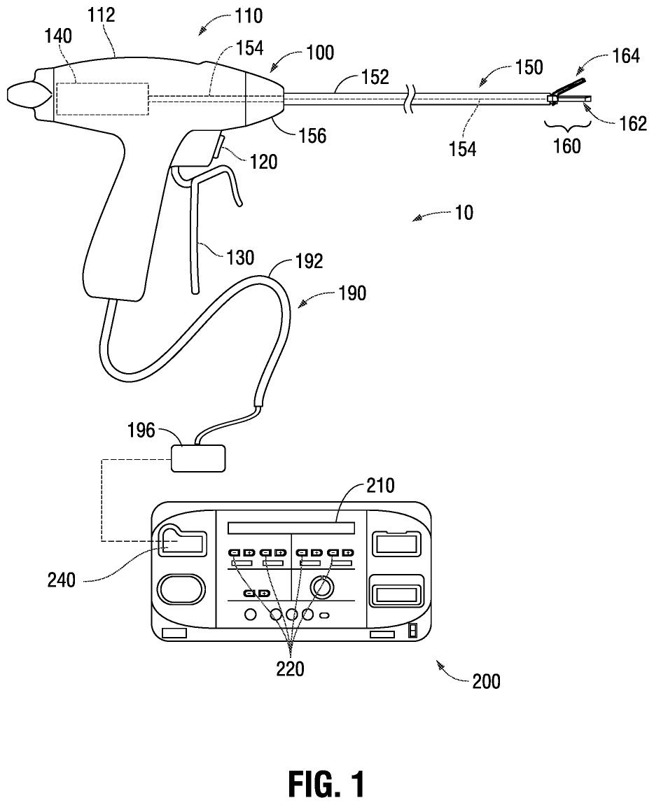

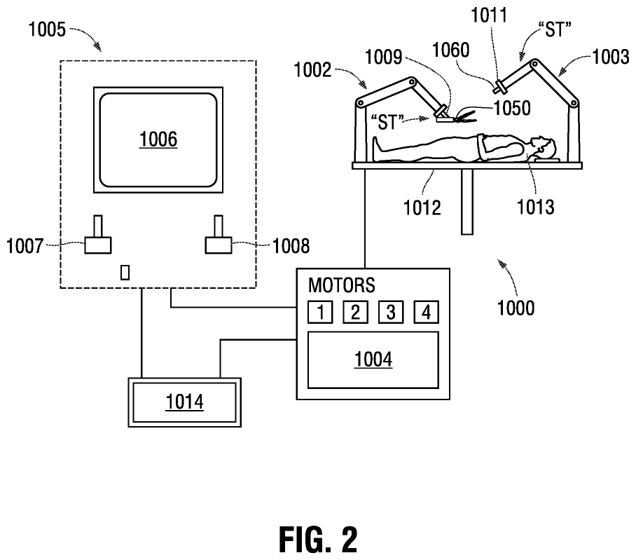

Referring to FIG. 1, a surgical system provided in accordance with aspects of the present disclosure is shown generally identified by reference numeral 10 including an ultrasonic surgical instrument 100 and a surgical generator 200. Ultrasonic surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200. As an alternative to handle assembly 110, surgical instrument 100 may include a robotic attachment housing for releasable engagement with a robotic arm of a robotic surgical system such as, for example, robotic surgical system 1000 (FIG. 2) detailed below.

Surgical generator 200 includes a display 210, a plurality user interface features 220, e.g., buttons, touch screens, switches, etc., and one or more plug ports including, for example, an ultrasonic plug port 240. A bipolar electrosurgical plug port, active and return monopolar electrosurgical plug ports, and/or other suitable plug ports are also contemplated. Surgical generator 200 is configured to produce ultrasonic drive signals for output through ultrasonic plug port 240 to ultrasonic surgical instrument 100 to activate ultrasonic surgical instrument 100 to deliver ultrasonic energy to tissue, as detailed below.

Continuing with reference to FIG. 1, handle assembly 110 includes a housing 112 defining a body portion and a fixed handle portion. Handle assembly 110 further includes an activation button 120 and a trigger 130. The body portion of housing 112 is configured to support an ultrasonic transducer 140. Ultrasonic transducer 140 may be permanently engaged with the body portion of housing 112 or removable therefrom. Ultrasonic transducer 140 includes a piezoelectric stack or other suitable ultrasonic transducer components electrically coupled to surgical generator 200, e.g., via one or more electrical lead wires (not shown), to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, e.g., to treat tissue therewith. An activation button 120 is disposed on housing 112 and coupled to or between ultrasonic transducer 140 and/or surgical generator 200, e.g., via one or more of the electrical lead wires, to enable activation of ultrasonic transducer 140 in response to depression of activation button 120. In some configurations, activation button 120 may include an ON/OFF switch. In other configurations, activation button 120 may include multiple actuation switches to enable activation from an OFF position to different actuated positions corresponding to different activation settings, e.g., a first actuated position corresponding to a LOW power activation setting and a second actuated position corresponding to a HIGH power activation setting. In still other configurations, separate activation buttons may be provided.

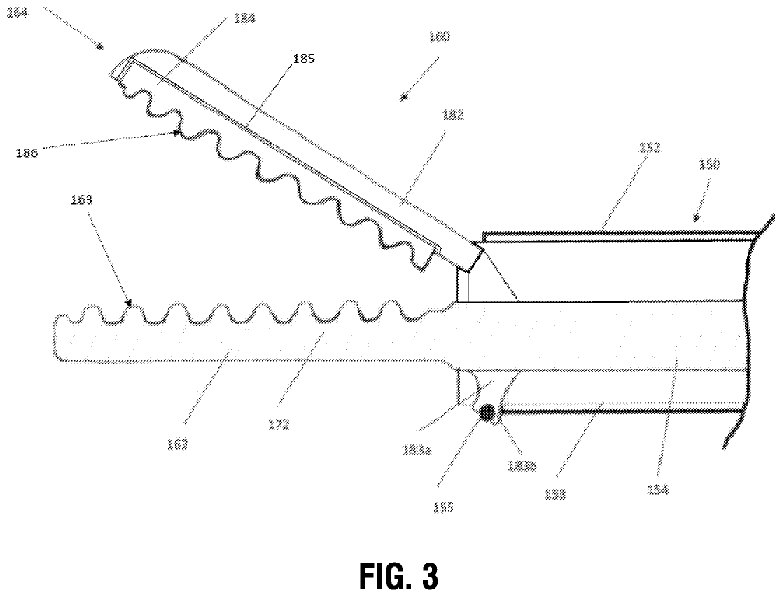

Elongated assembly 150 of surgical instrument 100 includes an outer drive sleeve 152, an inner support sleeve 153 (FIG. 3) disposed within outer drive sleeve 152, waveguide 154 extending through inner support sleeve 153 (FIG. 3), a drive assembly (not shown), a rotation knob 156, and end effector assembly 160 including blade 162 and a jaw member 164. Rotation knob 156 is rotatable in either direction to rotate elongated assembly 150 in either direction relative to handle assembly 110. The drive assembly operably couples a proximal portion of outer drive sleeve 152 to trigger 130 of handle assembly 110. A distal portion of outer drive sleeve 152 is operably coupled to jaw member 164 and a distal end of inner support sleeve 153 (FIG. 3) pivotably supports jaw member 164. As such, trigger 130 is selectively actuatable to thereby move outer drive sleeve 152 about inner support sleeve 153 (FIG. 3) to pivot jaw member 164 relative to blade 162 of end effector assembly 160 from a spaced apart position to an approximated position for grasping tissue between jaw member 164 and blade 162. The configuration of outer and inner sleeves 152, 153 (FIG. 3) may be reversed, e.g., wherein outer sleeve 152 is the support sleeve and inner sleeve 153 (FIG. 3) is the drive sleeve. Other suitable drive structures as opposed to a sleeve are also contemplated such as, for example, drive rods, drive cables, drive screws, etc.

Referring still to FIG. 1, the drive assembly may be tuned to provide a jaw clamping force, or jaw clamping force within a jaw clamping force range, to tissue grasped between jaw member 164 and blade 162 or may include a force limiting feature whereby the clamping force applied to tissue grasped between jaw member 164 and blade 162 is limited to a particular jaw clamping force or a jaw clamping force within a jaw clamping force range.

Waveguide 154, as noted above, extends from handle assembly 110 through the inner support sleeve. Waveguide 154 includes blade 162 disposed at a distal end thereof. Blade 162 may be integrally formed with waveguide 154, separately formed and subsequently attached (permanently or removably) to waveguide 154, or otherwise operably coupled with waveguide 154 to receive ultrasonic energy therefrom and to vibrate in response thereto, e.g., to heat and thereby treat tissue grasped between jaw member 164 and blade 162 or otherwise in contact with blade 162. Waveguide 154 and/or blade 162 may be formed from titanium, a titanium alloy, or other suitable electrically conductive material(s), although non-conductive materials are also contemplated. Waveguide 154 includes a proximal connector (not shown), e.g., a threaded male connector, configured for engagement, e.g., threaded engagement within a threaded female receiver of ultrasonic transducer 140, with ultrasonic transducer 140 such that ultrasonic motion produced by ultrasonic transducer 140 is transmitted along waveguide 154 to blade 162 for treating tissue grasped between blade 162 and jaw member 164 or positioned adjacent to blade 162.

Cable assembly 190 of surgical instrument 100 includes a cable 192 and an ultrasonic plug 196. Ultrasonic plug 196 is configured for connection with ultrasonic plug port 240 of surgical generator 200. The one or more electrical lead wires (not shown) electrically coupled to ultrasonic plug 196 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of activation button 120.

As an alternative to a remote generator 200, surgical system 10 may be at least partially cordless in that it incorporates an ultrasonic generator and/or a power source, e.g., a battery, thereon or therein. In this manner, the connections from ultrasonic surgical instrument 100 to external devices, e.g., a generator and/or power source, is reduced or eliminated.

With reference to FIG. 2, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be ultrasonic surgical instrument 100 (FIG. 1), wherein manual actuation features, e.g., actuation button 120 (FIG. 1), trigger 130 (FIG. 1), etc., are replaced with robotic inputs. In such configurations, robotic surgical system 1000 may include or be configured to connect to an ultrasonic generator, and/or a power source. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Referring to FIG. 3, end effector assembly 160 of ultrasonic surgical instrument 100 of surgical system 10 (FIG. 1) is detailed, although end effector assembly 160 may be utilized with any other suitable surgical instrument and/or surgical system. End effector assembly 160 includes a blade 162 and a jaw member 164. Blade 162 may define a linear configuration, may define a curved configuration, or may define any other suitable configuration along its length, e.g., including one or more straight, curved, and/or angled portions. With respect to curved and/or angled configurations, blade 162, more specifically, may be curved and/or angled in any direction relative to jaw member 164, for example, such that the distal tip of blade 162 is curved and/or angled towards jaw member 164, away from jaw member 164, or laterally (in either direction) relative to jaw member 164. Further, blade 162 may be formed to include multiple curves and/or angles in similar directions, multiple curves and/or angles in different directions within a single plane, and/or multiple curves and/or angles in different directions in different planes. In aspects, the curvatures and/or angles of jaw member 164 and blade 162 are complementary to one another such that jaw member 164 is aligned in apposition relative to blade 162 in an approximated position thereof.

Blade 162 may define a generally polygonal, rounded polygonal, or any other suitable cross-sectional configuration. Blade 162 further defines a tissue grasping surface 163 that generally opposes jaw member 164 and enables tissue to be grasped between blade 162 and jaw member 164 in the approximated position of jaw member 164. Tissue grasping surface 163 is described in greater detail below. In aspects, blade 162 is first formed via a first manufacturing process and tissue grasping surface 163 is defined within blade 162 in a second manufacturing process subsequent to the first. The manufacturing processes may be the same, e.g., machining, or may be different. Alternatively, blade 162 may be formed to include tissue grasping surface 163 via a single manufacturing process. Waveguide 154 or at least the portion of waveguide 154 proximally adjacent blade 162, may define a cylindrical shaped configuration. Plural tapered surfaces (not shown) may interconnect the cylindrically shaped waveguide 154 with the polygonal (rounded edge polygonal, or other suitable shape) configuration of blade 162 to define smooth transitions between the body of waveguide 154 and blade 162.

Blade 162 may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, IL, USA, or other suitable coatings and/or methods of applying coatings.

Continuing with reference to FIG. 3, jaw member 164 of end effector assembly 160 includes a more rigid structural body 182 and a more compliant jaw liner 184. Structural body 182 may be formed from or include a rigid metal, e.g., stainless steel, although other configurations are also contemplated. Structural body 182 includes a pair of proximal flanges 183a that are pivotably coupled to the inner support sleeve 153 via receipt of pivot bosses (not shown) of proximal flanges 183a within corresponding openings (not shown) defined within the inner support sleeve 153 and operably coupled with outer drive sleeve 152 via a drive pin 155 secured relative to outer drive sleeve 152 and pivotably received within apertures 183b defined within proximal flanges 183a. As such, sliding of outer drive sleeve 152 about inner support sleeve 153 pivots jaw member 164 relative to blade 162 from the spaced apart position to the approximated position to grasp tissue between jaw liner 184 of jaw member 164 and blade 162.

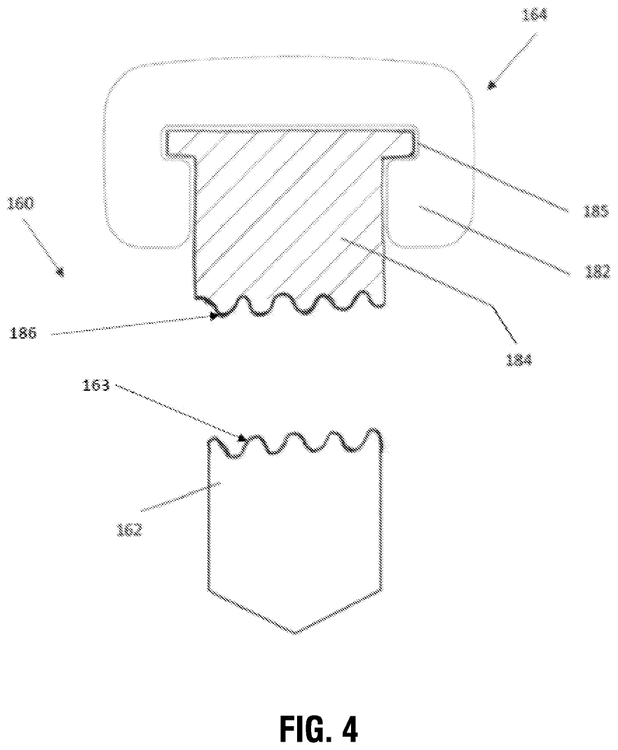

Jaw liner 184 is shaped complementary to a cavity 185 defined within structural body 182, e.g., defining a T-shaped configuration (see FIG. 4), to facilitate receipt and retention therein, although other configurations are also contemplated. Jaw liner 184 is fabricated from an electrically insulative, compliant material such as, for example, polytetrafluoroethylene (PTFE) or other suitable resilient polymeric material. The compliance of jaw liner 184 enables blade 162 to vibrate while in contact with jaw liner 184 without damaging components of ultrasonic surgical instrument 100 (FIG. 1) and without compromising the hold on tissue clamped between jaw member 164 and blade 162. Jaw liner 184 defines a tissue grasping surface 186 that generally opposes tissue grasping surface 163 of blade 162 in the approximated position of jaw member 164.

Tissue grasping surface 163 of blade 162 and tissue grasping surface 186 of jaw liner 184 define complementary configurations such that tissue grasping surfaces 163, 186 at least partially interfit within one another, e.g., wherein the ridges are at least partially received within the valleys, at a plurality of locations in the approximated position of jaw member 164. More specifically, in aspects, tissue grasping surfaces 163, 186 define substantially complementary wave-shaped configurations along at least portions of their lengths so as to at least partially interfit, in plural locations, along at least portions of the lengths thereof. The wave-shaped configurations may be formed via a plurality of alternating ridges and valleys extending transversely across tissue grasping surfaces 163, 186 or in any other suitable manner. Although tissue grasping surfaces 163, 186 are shown including rounded generally sine wave-shaped configurations in FIG. 3, other configurations are also contemplated such as, for example, square wave-shaped configurations, triangle wave-shaped configurations, sawtooth wave-shaped configurations, irregular and/or asymmetric wave-shaped configurations, combinations of any of the above, etc. Further, in aspects, the wave-shaped configurations may vary, e.g., in amplitude, frequency, and/or type along the lengths of tissue grasping surfaces 163, 186, continuously, discretely, in repeating patterns, etc. For example, distal end portions of tissue grasping surfaces 163, 186, may define more-pronounced and/or closer together ridges and more-recessed and/or closer together valleys, e.g., greater amplitudes and/or frequencies, respectively, as compared to more-proximal portions. Other configurations are also contemplated.

By providing substantially complementary wave-shaped configurations along at least portions of the lengths of tissue grasping surfaces 163, 186, tissue grasping is facilitated in the approximated position of jaw member 164. More specifically, with tissue grasped between tissue grasping surfaces 163, 186 of blade 162 and jaw liner 184, respectively, in the approximated position of jaw member 164, tissue grasping surfaces 163, 186 at least partially interfit with one another thus manipulating tissue to conform therebetween, thereby providing increased surface area for grasping the tissue and providing grasping force in multiple directions, all of which facilitate the grasping of tissue. Facilitating tissue grasping is particularly important in ultrasonic surgical instruments because of the manner in which they operate: wherein the blade is vibrating and the jaw liner is compliant to absorb some of the vibrating motion, which may result in tissue slippage or shifting in the absence of sufficient grasping capability.

Referring to FIG. 4, as an alternative to tissue grasping surfaces 163, 186 defining substantially complementary wave-shaped configurations along at least portions of their lengths (see FIG. 3), tissue grasping surfaces 163, 186 may define substantially complementary wave-shaped configurations across at least portions of their widths so as to interfit with one another at plural locations across at least portions of the widths thereof. The wave-shaped configurations may be formed via a plurality of alternating ridges and valleys extending longitudinally along portions or the entireties of tissue grasping surfaces 163, 186 or in any other suitable manner. Although tissue grasping surfaces 163, 186 are shown including rounded generally sine wave-shaped configurations in FIG. 4, other configurations are also contemplated such as any of those detailed above. Further, in aspects, tissue grasping surfaces 163, 186 may include longitudinally extending wave-shaped configurations (FIG. 3) on first portions thereof and transversely extending wave-shaped configurations (FIG. 4) on second portions thereof, e.g., wherein the first portions are proximal portions and the second portions are distal portions. The transversely extending wave-shaped configurations facilitate grasping of tissue similarly as detailed above.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. An ultrasonic surgical instrument, comprising:
an ultrasonic waveguide defining a blade at a distal end thereof, the blade defining a first tissue grasping surface, wherein the ultrasonic waveguide is configured to transmit ultrasonic energy therealong to the blade for treating tissue therewith; and
a jaw member movable relative to the blade from a spaced-apart position to an approximated position, the jaw member having a more-rigid structural body and a more-compliant jaw liner engaged with the structural body, the jaw liner defining a second tissue grasping surface having a configuration at least partially complementary to a configuration of the first tissue grasping surface,
wherein, in the approximated position, the first and second tissue grasping surfaces are configured to grasp tissue therebetween, the at least partially complementary configurations of the first and second tissue grasping surfaces facilitating tissue grasping.

2. The ultrasonic surgical instrument according to claim 1, wherein the first and second tissue grasping surfaces define complementary wave-shaped configurations.

3. The ultrasonic surgical instrument according to claim 2, wherein the first and second tissue grasping surfaces define complementary wave-shaped configurations extending longitudinally along portions of the blade and the jaw liner, respectively.

4. The ultrasonic surgical instrument according to claim 2, wherein the first and second tissue grasping surfaces define complementary wave-shaped configurations extending transversely across portions of the blade and the jaw liner, respectively.

5. The ultrasonic surgical instrument according to claim 2, wherein the first and second tissue grasping surfaces define complementary sine wave-shaped configurations.

6. The ultrasonic surgical instrument according to claim 1, wherein the first and second tissue grasping surfaces are configured to at least partially interfit with one another in the approximated position.

7. The ultrasonic surgical instrument according to claim 1, further comprising an ultrasonic transducer coupled to a proximal portion of the waveguide and configured to produce the ultrasonic energy for transmission along the waveguide to the blade.

8. The ultrasonic surgical instrument according to claim 7, further comprising a housing supporting the ultrasonic transducer.

9. The ultrasonic surgical instrument according to claim 8, further comprising a support member and a drive member extending distally from the housing, the waveguide extending through at least one of the support member or the drive member such that the blade extends distally therefrom, wherein the jaw member is pivotably supported by the support member and operably coupled to the drive member such that translation of the drive member pivots the jaw member between the spaced-apart and approximated positions.

10. The ultrasonic surgical instrument according to claim 9, further comprising a trigger extending from the housing and operably coupled to the drive member such that actuation of the trigger translates the drive member.
